(19) United States
(12) Patent Application Publication
(10) Pub. No.: US 2006/0134028 A1
(43) Pub. Date: Jun. 22, 2006

(54) Moisturizing compositions

(75) Inventor: Wei Hong Yu, Edison, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/266,307

(22) Filed: Nov. 4, 2005

Related U.S. Application Data
(60) Provisional application No. 60/624,543, filed on Nov. 4, 2004.

(51) Int. Cl. A61K 8/37

(52) U.S. Cl. 424/59

(57) ABSTRACT

The present invention relates to compositions useful for moisturizing keratinous materials which contain at least one polyol and a solid wax capable of stabilizing the at least one polyol in an anhydrous solid composition.

### BACKGROUND OF THE INVENTION

1. Field of the Invention

The present invention relates to compositions useful for moisturizing keratinous materials which contain at least one polyol and at least one solid wax capable of stabilizing the at least one polyol in an anhydrous solid composition, for example, a solid wax having a hydroxyl value ranging from about 38 to about 48.

2. Description of the Background

Moisturizing compositions are known and widely used in the art. Their applicability ranges from providing moisture to lips, skin, hair, and/or nails as a treatment when they are dry or as a preventative or for the maintenance of the lips, skin, hair and/or nails as a part of an individual's routine regimen of hygiene. For example, lip care compositions are commonly used not only to provide a make-up effect, e.g., color, but also to protect, soothe and/or moisturize. Polyols, such as for example, glycerin are known humectants which provide a moisture benefit to the lips, skin, hair, and/or nails when applied. However, polyols, such as for example, glycerin are not particularly stable in those compositions, i.e., they leech out of the composition.

Therefore, there remains a need to stabilize glycerin thereby providing additional moisturizing compositions that provide an exceptional moisturizing effect with little or no adverse effects, such as irritation, when applied.

### SUMMARY OF THE INVENTION

The inventors have discovered that exceptional moisturizing benefits can be achieved on keratinous material by applying a composition combining at least one polyol and at least one solid wax capable of stabilizing the at least one polyol in an anhydrous solid composition, for example, a solid wax having a hydroxyl value ranging from about 38 to about 48. In addition, the inventors have discovered that the addition of the at least one wax stabilizes the composition with at least one polyol.

Thus, the present invention provides a composition comprising moisturizing effective amounts of at least one polyol and at least one solid wax capable of stabilizing the at least one polyol in an anhydrous solid composition.

The invention also provides methods of providing a moisturizing benefit to an individual with the composition.

The invention also provides methods of making the composition.

### DETAILED DESCRIPTION OF THE INVENTION

The invention, in one embodiment, relates to cosmetic, dermatological, and pharmaceutical products containing a composition having moisturizing effective amounts of at least one polyol and at least one solid wax capable of stabilizing the at least one polyol in an solid composition. In a preferred embodiment, the composition is anhydrous.

The polyols used in the present invention are polyols that are used as humectants and/or moisturizers in skin care compositions. Examples include hydrophilic and comprise polyhydric alcohols, ethoxylated and propoxylated polyols, polysaccharides, and mixtures thereof. Preferred polyols/moisturizers are selected from glycerin, panthenol, hexylene glycol, propylene glycol, butylene glycol, alkanediols, polyethylene glycol, polypropylene glycol, sorbitol, and mixtures thereof. Most preferred is glycerin.

The polyol is incorporated into the composition such that when in combination with the solid wax it provides a moisturizing benefit to the skin, lips and/or keratinous material of the user greater than either the polyol or solid wax separately. This combination in a composition provides a moisturizing effect, which means an increase in perceived moisture on the lips, skin, hair or other keratinous materials after the composition of the invention is applied thereto. Without being limited to theory, it is believed that the hydroxyl groups of the solid wax interact with glycerin thereby increasing the stability of glycerin in the composition and ultimately provides better moisturizing effects.

In another embodiment, moisture can be determined empirically using devices designed to measure moisture content, such as the SkinChip® sensor (L'Oréal and STMicroelectronics) or Novameter™ (NovaTechnology, Gloucester, Mass.). Using such a moisture testing device, the keratinous material onto which the composition described herein has been applied can benefit from at least a 5% increase in moisture relative to the moisture prior to application, which includes at least 10%, 15%, 20%, 25%, 30%, 40%, 50%, all subranges and values there between, or more.

For example, the polyol may be present in amounts from 0.5 to 75% by weight of the composition, inclusive of 1, 5, 10, 15, 20, 25, 30, 35, 40, 45, 50, 55, 60, 65, and 70% by weight of the composition, and all ranges and sub ranges there between. In a preferred embodiment, the polyol is present in an amount from about 1 to about 5%, including 2%, 3%, 4% and all ranges and subranges there between.

The solid wax capable of stabilizing the at least one polyol in an anhydrous solid composition, for example, is a solid wax having a hydroxyl value ranging from about 10 to about 70, including from about 15 to about 60, including from about 30 to about 50, including from about 38 to about 48, which also includes values of 15, 20, 25, 35, 40, 45, 55, 65 and all values and subranges there between. In one embodiment, the solid wax can be a hydroxy polyester wax which is derived from the natural fatty acids and synthetic fatty alcohols yielding a wax that mimics the complex polyester fraction of natural beeswax which gives beeswax its unique plastic properties. In a generally preferred example, the wax should be solid at room temperature (about 25° C.). For example, the waxes that are similar to natural beeswax are described in International Cosmetic Ingredient Dictionary and Handbook Vol. 2 (7^{th} ed. 1999) and in EP 0887069 and U.S. Pat. No. 3,914,131, the contents of which are incorporated herein, may be used.

The stability of the at least one polyol when combined with the solid wax described herein can be measured visually after preparation of the composition. For example, in an anhydrous solid composition, such as a stick composition, an increase in stability is visually presented as a decrease in sweating on the surface of the stick or the number of droplets formed on the stick's surface. While increase and decrease are relative terms, one of skill in the art will recognize that the relevant comparison is made to a similar stick but formulated without the wax or different concentrations of the wax. In a preferred embodiment, the combination of the at least one solid wax and the at least one polyol results in a composition that does not exhibit any sweating or the appearance of droplets on the solid stick formulation.

In a preferred embodiment, the wax is the wax sold as Kester K82P (Koster Keunen, Inc.), which is made up of a range of hydroxyl polyesters with an average chain length of 66 carbon atoms, C20-40 alkyl hydroxystearyl stearate (CAS 151661-95-9).

The at least one solid wax is incorporated into the composition such that when it is in combination with the at least one polyol, the composition provides a moisturizing benefit to the skin, lips and/or keratinous material of the user greater than either the polyol or solid wax separately. For example, the wax may be present in amounts from 0.5 to 75% by weight of the composition, inclusive of 1, 2, 3, 4, 5, 6, 7, 8, 9, 10, 15, 20, 25, 30, 35, 40, 45, 50, 55, 60, 65, and 70% by weight of the composition, and all ranges and sub ranges there between.

The invention applies not only to make-up products for at least one keratinous material such as lip compositions, lip pencils, foundations including foundations which may be cast in the form of a stick or a dish, concealer products, temporary tattoo products, eyeliners, mascara bars but also to body hygiene products such as deodorant sticks, and to care products and products for treating at least one keratinous material such as sunscreen and after-sun products which may be in stick form. The present invention may be in the form of mascara product including mascara bars, an eyeliner product, a foundation product, a lipstick product, a blush for cheeks or eyelids, a deodorant product, a make-up product for the body, a make-up-removing product, an eyeshadow product, a face powder product, a concealer product, a treating shampoo product, a hair conditioning product, a sun screen, colorant for the skin or hair, or skin care formula such as, for example, anti-pimple or shaving cut formulas. As defined herein, a deodorant product is a body hygiene product and does not relate to care, make-up, or treatment of keratin materials, including keratin fibers, skin, or lips.

For example, the composition of the present invention may be in a form chosen from a paste, a solid, a gel, and a cream. It may be an emulsion, such as an oil-in-water or water-in-oil emulsion, a multiple emulsion, such as an oil-in-water-in-oil emulsion or a water-in-oil-in-water emulsion, or a solid, rigid or supple gel, including anhydrous gels. In one embodiment, the composition of the invention is anhydrous. The composition of the invention may, for example, comprise an external or continuous fatty phase. In another embodiment, the composition of the invention is transparent or clear, including for example, a composition without pigments. The composition can also be in a form chosen from a translucent anhydrous gel and a transparent anhydrous gel. The composition can also be a molded composition or cast as a stick or a dish. The composition in one embodiment is a solid such as a molded stick or a poured stick.

The compositions may also contain one or more film-forming polymers.

The film-forming polymer of the compositions may be chosen from any silicone resin that has film forming properties and any other organic material with film forming properties.

The film forming polymer of the compositions according to the present invention may also be a non silicone film former. This non silicone film former may be chosen from, for example, polyethylene; vinylpyrrolidone/vinyl acetate (PVP/VA) copolymers such as the Luviskol® VA grades (all ranges) from BASF® Corporation and the PVP/VA series from ISP; acrylic fluorinated emulsion film formers including Foraperle® film formers such as Foraperle® 303 D from Elf Atochem (although Foraperle® may not be appropriate for some cosmetic formulations); GANEX® copolymers such as butylated PVP, PVP/Hexadecene copolymer, PVP/Eicosene copolymer or tricontanyl;

Poly(vinylpyrrolidone/diethylaminoethyl methacrylate) or

PVP/Dimethylaminoethylmethacrylate copolymers such as Copolymer 845; Resin ACO-5014 (Imidized IB/MA copolymer); other PVP based polymers and copolymers; alkyl cycloalkylacrylate copolymers (See WO 98/42298, the disclosure of which is hereby incorporated by reference); Mexomere® film formers and other allyl stearate/vinyl acetate copolymers (allyl stearate/VA copolymers); polyolprepolymers such as PPG-12/SMDI copolymer, polyolprepolymers such as PPG-12/SMDI copolymer, Poly(oxy-1,2-ethanediyl), α-hydro-ω-hydroxy-polymer with 1,1′-methylene-bis-(4-isocyanatocyclohexane) available from Barnet; Avalure™ AC Polymers (Acrylates Copolymer) and Avalure™ UR polymers (Polyurethane Dispersions), available from BFGoodrich.

The film former which also may be used within the framework of the invention includes film formers having any film former chemistry known in the art such as: PVP, acrylates, and urethanes; synthetic polymers of the polycondensate type or free-radical type, or ionic type, polymers of natural origin and mixtures thereof or any other film former known within the practice of the cosmetic and pharmaceutical arts which one skilled in the art may determine to be compatible. Film formers that may be used are also disclosed in the International Cosmetic Ingredient Dictionary and Handbook Vol. 2 (7^{th} ed. 1999), more particularly the emollients disclosed on pages 1636-1638. The disclosure of the International Cosmetic Ingredient Dictionary and Handbook Vol. 2, pages 1636-1638, is hereby incorporated by reference.

An appropriate concentration of the film former may be determined by one of skill in the art and can vary considerably based on the application. For example, for cosmetic compositions, the film former may be used in an amount from 0.1% to 20% such as, for example, from 1% to 10% by weight, relative to the total weight of the composition.

Depending on the intended application, such as a stick, hardness of the composition may also be considered. The hardness of a composition may, for example, be expressed in grams (g). The composition of the present invention may, for example, have a hardness ranging from 20 g to 2000 g, such as from 20 g to 900 g, and further such as from 20 g to 600 g.

This hardness is measured in one of two ways. A first test for hardness is according to a method of penetrating a probe into said composition and in particular using a texture analyzer (for example TA-XT2 from Rhéo) equipped with an ebonite cylinder of height 25 mm and diameter 8 mm. The hardness measurement is carried out at 20° C. at the center of 5 samples of said composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 g.

The second test for hardness is the “cheese wire” method, which involves cutting an 8.1 mm tube of composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 g to 300 g, such as from 30 g to 250 g, and further such as from 30 g to 200 g.

The hardness of the composition of the present invention may be such that the compositions are self-supporting and can easily disintegrate to form a satisfactory deposit on a keratinous material. In addition, this hardness may impart good impact strength to the inventive compositions which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within the scope of the invention.

According to the present invention, the compositions in stick form may also possess the properties of deformable, flexible elastic solids and may also have noteworthy elastic softness upon application to a keratinous material.

The compositions of the present invention may also contain one or more volatile oils or solvents. For the purposes of the invention, the expression “volatile solvent or oil” means any non-aqueous medium capable of evaporating on contact with the skin or the lips in less than one hour at room temperature and atmospheric pressure. The volatile solvent(s) of the invention is(are) organic solvents, such as volatile cosmetic oils that are liquid at room temperature, having a non-zero vapor pressure, at room temperature and atmospheric pressure, ranging in particular from 10⁻² to 300 mmHg and, for example, greater than 0.3 mmHg. The expression “non-volatile oil” means an oil which remains on the skin or the lips at room temperature and atmospheric pressure for at least several hours, such as those having a vapor pressure of less than 10⁻² mmHg.

According to the invention, these volatile solvents may facilitate the staying power or long wearing properties of the composition on the skin, the lips or superficial body growths. The solvents can be chosen from hydrocarbon-based solvents, silicone solvents optionally comprising alkyl or alkoxy groups that are pendant or at the end of a silicone chain, and a mixture of these solvents.

The volatile oil(s), in one embodiment, is present in an amount ranging from 0% to 95.5% relative to the total weight of the composition, such as from 2% to 75% or, for example, from 10% to 45%. This amount will be adapted by a person skilled in the art according to the desired staying power or long wearing properties.

The compositions of the present invention may also contain one or more sun screening agents.

Sunscreens according to this invention which are chemical absorbers actually absorb harmful ultraviolet radiation. It is well known that chemical absorbers are classified, depending on the type of radiation they protect against, as either UV-A or UV-B absorbers. UV-A absorbers generally absorb radiation in the 320 to 400 nm region of the ultraviolet spectrum. UV-A absorbers include anthranilates, benzophenones, and dibenzoyl methanes. UV-B absorbers generally absorb radiation in the 280 to 320 nm region of the ultraviolet spectrum. UV-B absorbers include p-aminobenzoic acid derivatives, camphor derivatives, cinnamates, and salicylates.

Classifying the chemical absorbers generally as UV-A or UV-B absorbers is accepted within the industry. However, a more precise classification is one based upon the chemical properties of the sunscreens. There are eight major classifications of sunscreen chemical properties which are discussed at length in “Sunscreens—Development, Evaluation and Regulatory Aspects,” by N. Shaath et al., 2nd. Edition, pages 269-273, Marcel Dekker, Inc. (1997). This discussion, in its entirety, is incorporated by reference herein.

The sunscreens which may be formulated according to the present invention typically comprise chemical absorbers, but may also comprise physical blockers. Exemplary sunscreens which may be formulated into the compositions of the present invention are chemical absorbers such as p-aminobenzoic acid derivatives, anthranilates, benzophenones, camphor derivatives, cinnamic derivatives, dibenzoyl methanes (such as avobenzone also known as Parsol®1789), diphenylacrylate derivatives, salicylic derivatives, triazine derivatives, benzimidazole compounds, bis-benzoazolyl derivatives, methylene bis-(hydroxyphenylbenzotriazole) compounds, the sunscreen polymers and silicones, or mixtures thereof. These are variously described in U.S. Pat. Nos. 2,463,264, 4,367,390, 5,166,355 and 5,237,071 and in EP-0,863,145, EP-0,517,104, EP-0,570,838, EP-0,796,851, EP-0,775,698, EP-0,878,469, EP-0,933,376, EP-0,893,119, EP-0,669,323, GB-2,303,549, DE-1,972,184 and WO-93/04665, also expressly incorporated by reference. Also exemplary of the sunscreens which may be formulated into the compositions of this invention are physical blockers such as cerium oxides, chromium oxides, cobalt oxides, iron oxides, red petrolatum, silicone-treated titanium dioxide, titanium dioxide, zinc oxide, and/or zirconium oxide, or mixtures thereof.

A wide variety of sunscreens is described in U.S. Pat. No. 5,087,445, issued to Haffey et al. on Feb. 11, 1992; U.S. Pat. No. 5,073,372, issued to Turner et al. on Dec. 17, 1991; and Chapter VIII of Cosmetics and Science and Technology by Segarin et al., pages 189 et seq. (1957), all of which are incorporated herein by reference in their entirety.

Sunscreens which may be formulated into the compositions of the instant invention are those selected from among: aminobenzoic acid, amyldimethyl PABA, cinoxate, diethanolamine p-methoxycinnamate, digalloyl trioleate, dioxybenzone, 2-ethoxyethyl p-methoxycinnamate, ethyl 4-bis(hydroxypropyl)aminobenzoate, 2-ethylhexyl-2-cyano-3,3-diphenylacrylate, ethylhexyl p-methoxycinnamate, 2-ethylhexyl salicylate, glyceryl aminobenzoate, homomenthyl salicylate, homosalate, 3-imidazol-4-ylacrylic acid and ethyl ester, methyl anthranilate, octyldimethyl PABA, 2-phenylbenzimidazole-5-sulfonic acid and salts, red petrolatum, sulisobenzone, titanium dioxide, triethanolamine salicylate, N,N,N-trimethyl-4-(2-oxoborn-3-ylidene methyl)anillinium methyl sulfate, and mixtures thereof.

Sunscreens active in the UV-A and/or UV-B range can also include: p-aminobenzoic acid, oxyethylene (25 mol) p-aminobenzoate, 2-ethylhexyl p-dimethylaminobenzoate, ethyl N-oxypropylene p-aminobenzoate, glycerol p-aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammunium)-benzyliden-bornan-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′dimethoxybenzophenone, 2-hydroxy-4-n-octoxybenzophenone, 2-hydroxy-4-methoxy-4′-methoxybenzophenone, -(2-oxoborn-3-ylidene)-tolyl-4-sulfonic acid and soluble salts thereof, 3-(4′-sulfo)benzyliden-bornan-2-one and soluble salts thereof, 3-(4′methylbenzylidene)-d,1-camphor, 3-benzylidene-d,1-camphor, benzene 1,4-di(3-methylidene-10-camphosulfonic) acid and salts thereof (the product Mexoryl SX described in U.S. Pat. No. 4,585,597 issued to Lange et al. on Apr. 29, 1986), urocanic acid, 2,4,6-tris[p-(2′-ethylhexyl-1′-oxycarbonyl)-anilino]-1,3,5-triazine, 2-[(p-(tertiobutylamido)anilino]-4,6-bis-[(p-(2′-ethylhexyl-1′-oxycarbonyl)anilino]-1,3,5-triazine, 2,4-bis{[4-(2-ethyl-hexyloxy)]-2-hydroxyl-phenyl}-6-(4-methoxy-phenyl)-1,3,5-triazine (“TINOSORB S” marketed by Ciba), the polymer of N-(2 et 4)-[(2-oxoborn-3-yliden)methyl]benzyl]-acrylamide, 1,4-bisbenzimidazolyl-phenylen-3,3′,5,5′-tetrasulfonic acid and salts thereof, the benzalmalonate-substituted polyorganosiloxanes, the benzotriazole-substituted polyorganosiloxanes (Drometrizole Trisiloxane), dispersed 2,2′-methylene-bis-[6-(2H-benzotriazol-2-yl)-4-(1,1,3,3-tetramethylbutyl)phenol] such as that marketed under the trademark MIXXIM BB/100 by Fairmount Chemical, or micronized in dispersed form thereof such as that marketed under the trademark TINOSORB M by Ciba-Geigy, and solubilized 2,2′-methylene-bis-[6-(2H-benzotriazol-2-yl)-4-(methyl)phenol] such as that marketed under the trademark MIXXIM BB/200 by Fairmount Chemical. Typically combinations of one of more of these sunscreens are used.

The dibenzoyl methane derivatives other than avobenzone are described, for example, in FR-2,326,405, FR-2,440,933 and EP-0,114,607, hereby expressly incorporated by reference.

Other dibenzoyl methane sunscreens other than avobenzone include (whether singly or in any combination): 2-methyldibenzoylmethane; 4-methyldibenzoylmethane; 4-isopropyldibenzoylmethane; 4-tert.-butyldibenzoylmethane; 2,4-dimethyldibenzoylmethane; 2,5-dimethyldibenzoylmethane; 4,4′-diisopropyldibenzoylmethane; 4,4′-dimethoxydibenzoylmethane; 2-methyl-5-isopropyl-4′-methoxydibenzoylmethane; 2-methyl-5-tert.-butyl-4′-methoxydibenzoylmethane; 2,4-dimethyl-4′-methoxydibenzoylmethane; 2,6-dimethyl-4-tert.-butyl-4′-methoxydibenzoylmethane; Additional sunscreens that can be used are described in pages 2954-2955 of the International Cosmetic Ingredient Dictionary and Handbook (9^{th} ed. 2002).

The compositions of the invention may further comprise at least one additional fatty material. The at least one additional fatty material may, for example, be chosen from gums, fatty materials pasty at ambient temperature, and resins.

The compositions of the invention may further include formulation aids which are usually employed in the field of application envisaged. The formulation aids used in the present invention can be, but are not limited to, fatty substances. Useful fatty substances include, but are not limited to, organic and organosilicone emulsifiers for water-in-oil systems. Examples of organic emulsifiers include any ethoxylated surfactants known in the art such as Polysorbate-20, Laureth-7, Laureth-4, Sepigel® 305 available from SEPPIC and other similar ingredients disclosed in the International Cosmetic Ingredient Dictionary and Handbook Vol. 4 (9^{th} ed. 2002), more particularly the emulsifiers disclosed on pages 2962-2971. The disclosure of the International Cosmetic Ingredient Dictionary and Handbook Vol. 4, pages 2962-2971, is hereby incorporated by reference. Examples of organosilicone emulsifiers include cetyl dimethicone copolyol-polyglyceryl-4-isostearate-hexylaurate (ABIL® WE 09) available from Goldschmidt Chemical Corporation, Cetyl Dimethicone Copolyol (ABIL® EM 90), (ABIL® EM 97), Laurylmethicone Copolyol (5200), Cyclomethicone (and) Dimethicone Copolyol (DC 5225 C and DC 3225 C) available from GE Silicones, Cyclopentasiloxane & Dimethicone Copolyol (GE SF 1528) or any other formulation aids known by one of skill in the art. Other fatty substances useful as formulation aids include but are not limited to, silicones in esterified or unesterified liquid form or in esterified solid form, such as behenate dimethicone; and non-silicone fatty substances including oils such as vegetable and mineral oil; animal and/or synthetic waxes such as beeswax, parafin, rice bran wax, candelilla wax, carnauba wax and derivatives thereof, and hydrocarbon gels or bentone type gels, such as Gel SS71, Gel EA2786, Quaternium-18 Bentonite, or 38 CE.

These substances may be selected variously by the person skilled in the art in order to prepare a composition which has the desired properties, for example, consistency or texture.

Plasticizers may also be added to the compositions to improve the flexibility and cosmetic properties of the resulting formulation. Plasticizers are materials which soften synthetic polymers. They are frequently required to avoid brittleness and cracking of film formers. One skilled in the art may routinely vary the amount of plasticizer desired based on the properties desired and the application envisaged. Plasticizers useful in the practice of the invention include lecithin, polysorbates, dimethicone copolyol, glycols, citrate esters, glycerin, dimethicone, and other similar ingredients disclosed in the International Cosmetic Ingredient Dictionary and Handbook Vol. 4 (9^{th} ed. 2002), more particularly the plasticizers disclosed on page 2927. The disclosure of the International Cosmetic Ingredient Dictionary and Handbook Vol. 4, page 2927, is hereby incorporated by reference.

The composition of the present invention may also further comprise at least one suitable additive commonly used in the field concerned chosen from coloring agents, antioxidants, essential oils, preserving agents, fragrances, fillers, pasty fatty substances, waxy fatty substances, neutralizing agents, liposoluble polymers, and cosmetically active agents and dermatological active agents such as, for example, emollients, moisturizers, vitamins and essential fatty acids. The compositions of the invention may also be optionally thickened with an aqueous-phase thickener or gelled with a gelling agent and/or containing ingredients soluble in water.

In addition to the solid wax, e.g., hydroxyl polyester wax, the compositions may also contain one or more additional waxes, synthetic or natural. As used herein, a “wax” may be any lipophilic fatty compound. Non-limiting examples of such waxes include waxes of natural origin, such as beeswax, carnauba wax, candelilla wax, ouricury wax, Japan wax, cork fiber wax, sugar cane wax, paraffin waxes, lignite wax, microcrystalline waxes, lanolin wax, montan wax and ozokerites, hydrogenated oils such as hydrogenated jojoba oil, jojoba esters, waxes of synthetic origin, such as polyethylene waxes derived from polymerization of ethylene, waxes obtained by Fischer-Tropsch synthesis, fatty acid esters and glycerides, and silicone waxes such as derivatives of poly(di)methylsiloxane.

Another embodiment of the invention relates to a skin lip, or keratinous material care or make-up composition comprising the components of the composition as described herein.

Another embodiment of the invention relates to increasing the hydration properties of at least one polyol in a composition by adding the at least one solid wax to the composition comprising at least one polyol.

Additionally, an embodiment of the invention relates to a method of providing a moisturizing benefit to the skin, lips, or keratinous materials by applying to such skin, lips, and/or keratinous material in need thereof the compositions as described herein such that the skin, lips, and/or keratinous material become moisturized.

In the method of providing a moisturizing benefit, the composition can be applied to dry skin, lips and/or keratinous material as a treatment, the composition can be applied prior to any visible detection of dryness occurs as a preventative, or the composition can be incorporated into an individual's routine hygiene and healthcare maintenance regimen.

The present invention also envisages kits and/or prepackaged materials suitable for consumer use containing one or more compositions according to the description herein to provide a moisturizing benefit to the skin, lips, and/or keratinous material. The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

In another embodiment, the present invention is directed to a process of making a moisturizing composition with glycerin and the wax, the process including a method of mixing the two ingredients directly or first mixing separately with additional compositional components described herein and then mixing the ingredients together.

### EXAMPLES

### Example 1

### A Lipstick Composition

| Phase | Component | Amount |
|---|---|---|
| A | Hydrogenated polyisobutene | 15.86 |
| | Preservative | 0.04 |
| | Caprylic/Capric/Stearic Triglyceride | 8.10 |
| | Bis diglyceryl polyacyladipate-2 | 12.00 |
| | Diisostearyl malate | 5.00 |
| | Triisononanoin | 7.8 |
| | Tridecyl Trimellilate | 10.00 |
| | PVP/Hexadecene Copolymer | 4.00 |
| | glycerin | 1.00 |
| B | Polyethylene | 8.50 |
| | Hydrogenated coco-glycerides | 5.00 |
| | Synthetic wax (Kester K82P) | 1.00 |
| C | Silica | 1.00 |
| | Diisostearyl Malate | 10.00 |
| | Pigments | 8.20 |
| | cornstarch/acrylamide/Sodium acrylate copolymer | 0.5 |
| D | Active Phase | 2.00 |

The components of each phase can be mixed separately and then mixed together using the appropriate mixers. The resultant composition can then be formed into a stick and will provide exceptional moisturizing effect to the lips upon application.

### Example 2

### A Lipstick Composition

| Phase | Component | Amount |
|---|---|---|
| A | Hydrogenated polyisobutene | 15.86 |
| | Preservative | 0.04 |
| | Caprylic/Capric/Stearic Triglyceride | 8.10 |
| | Bis-diglyceryl polyacyladipate-2 | 12.00 |
| | Diisostearyl malate | 5.00 |
| | Triisononanoin | 7.8 |
| | Tridecyl Trimellilate | 10.00 |
| | PVP/Hexadecene Copolymer | 3.00 |
| | glycerin | 2.00 |
| B | Polyethylene | 8.50 |
| | Hydrogenated coco-glycerides | 5.00 |
| | Synthetic wax (Kester K82P) | 1.00 |
| C | Silica | 1.00 |
| | Diisostearyl Malate | 10.00 |
| | Pigments | 6.70 |
| | cornstarch/acrylamide/Sodium acrylate copolymer | 2.0 |
| D | Active Phase | 2.00 |

The components of each phase can be mixed separately and then mixed together using the appropriate mixers. The resultant composition can then be formed into a stick and will provide exceptional moisturizing effect to the lips upon application.

Obviously, numerous modifications and variations of the present invention are possible in light of the above teachings. It is therefore to be understood that within the scope of the appended claims, the invention may be practiced otherwise than as specifically described herein.

1. A composition suitable for application to keratinous material, comprising moisturizing effective amounts of at least one polyol and at least one wax, which is solid at about 25° C., and has a hydroxyl value of from 10 to 70.

2. The composition of claim 1, which is anhydrous.

3. The composition of claim 1, further comprising one or more sunscreen agents.

4. The composition of claim 1, which is in the form of a cream, gel, lotion, or stick.

5. The composition of claim 4, which is in the form of a stick.

6. The composition of claim 1, which further comprises one or more film-forming polymers.

7. The composition of claim 1, which further comprises one or more pigments.

8. The composition of claim 1, wherein the at least one polyol is present in an amount from 0.5 to 75% by weight of the composition.

9. The composition of claim 8, wherein the at least one polyol is present in an amount from 1 to 5% by weight of the composition.

10. The composition of claim 1, wherein the at least one wax is present in an amount from 0.5 to 75% by weight of the composition.

11. The composition of claim 10, wherein the at least one wax is present in an amount from 1 to 5% by weight of the composition.

12. The composition of claim 1, wherein the at least one wax is a hydroxyl polyester wax.

13. The composition of claim 1, wherein the at least one polyol is glycerin.

14. The composition of claim 1, wherein the at least one wax is at least one hydroxyl polyester wax and the at least one polyol is glycerin.

15. The composition of claim 14, which is anhydrous.

16. The composition of claim 14, further comprising one or more sunscreen agents.

17. The composition of claim 14, which is in the form of a cream, gel, lotion, or stick.

18. The composition of claim 17, which is in the form of a stick.

19. The composition of claim 14, which further comprises one or more film-forming polymers.

20. The composition of claim 14, which further comprises one or more pigments.

21. The composition of claim 14, wherein the glycerin is present in an amount from 0.5 to 75% by weight of the composition.

22. The composition of claim 21, wherein the glycerin is present in an amount from 1 to 5% by weight of the composition.

23. The composition of claim 14, wherein the at least one hydroxyl polyester wax is present in an amount from 0.5 to 75% by weight of the composition.

24. The composition of claim 23, wherein the at least one hydroxyl polyester wax is present in an amount from 1 to 5% by weight of the composition.

25. A method of providing a moisturizing benefit to one or more keratinous materials, comprising applying a composition comprising moisturizing effective amounts of at least one polyol and at least one wax, which is solid at about 25° C., and has a hydroxyl value of from 10 to 70, in an amount effective to provide a moisturizing benefit thereto.

26. The method of claim 25, wherein the keratinous material is skin.

27. The method of claim 25, wherein the keratinous material is the lips.

28. The method of claim 25, wherein the composition is anhydrous.

29. The method of claim 25, wherein the composition further comprises one or more sunscreen agents.

30. The method of claim 25, wherein the composition is in the form of a cream, gel, lotion, or stick.

31. The method of claim 30, wherein the composition is in the form of a stick.

32. The method of claim 25, wherein the composition further comprises one or more film-forming polymers.

33. The method of claim 25, wherein the composition further comprises one or more pigments.

34. The method of claim 25, wherein the at least one polyol is present in an amount from 0.5 to 75% by weight of the composition.

35. The method of claim 34, wherein the at least one polyol is present in an amount from 1 to 5% by weight of the composition.

36. The method of claim 25, wherein the at least one wax is present in an amount from 0.5 to 75% by weight of the composition.

37. The method of claim 36, wherein the at least one wax is present in an amount from 1 to 5% by weight of the composition.

38. The method of claim 25, wherein the at least one wax is a hydroxyl polyester wax.

39. The method of claim 25, wherein the at least one polyol is glycerin.

40. The method of claim 25, wherein the at least one wax is at least one hydroxyl polyester wax and the at least one polyol is glycerin.

41. The method of claim 40, wherein the keratinous material is skin.

42. The method of claim 40, wherein the keratinous material is the lips.

43. The method of claim 40, wherein the composition is anhydrous.

44. The method of claim 40, wherein the composition further comprises one or more sunscreen agents.

45. The method of claim 40, wherein the composition is in the form of a cream, gel, lotion, or stick.

46. The method of claim 45, wherein the composition is in the form of a stick.

47. The method of claim 40, wherein the composition further comprises one or more film-forming polymers.

48. The method of claim 40, wherein the composition further comprises one or more pigments.

49. The method of claim 40, wherein the glycerin is present in an amount from 0.5 to 75% by weight of the composition.

50. The method of claim 49, wherein the glycerin is present in an amount from 1 to 5% by weight of the composition.

51. The method of claim 40, wherein the at least one hydroxyl polyester wax is present in an amount from 0.5 to 75% by weight of the composition.

52. The method of claim 51, wherein the at least one hydroxyl polyester wax is present in an amount from 1 to 5% by weight of the composition.

53. A method of manufacturing the composition of claim 1, comprising mixing the at least one polyol with the at least one wax.

54. A method of manufacturing the composition of claim 14, comprising mixing the at least one polyol with the at least one wax.
